(19) United States
(12) Patent Application Publication
(10) Pub. No.: US 2022/0233770 A1
(43) Pub. Date: Jul. 28, 2022

(54) Automatic monitoring of fluid injection procedures using a sensing catheter

(71) Applicant: The Board of Trustees of the Leland Stanford Junior University, Stanford, CA (US)

(72) Inventors: Alexander Michael Vezeridis, Los Altos, CA (US); Bruce L. Daniel, Stanford, CA (US); Lawrence V. Hofmann, Stanford, CA (US)

(21) Appl. No.: 17/582,677

(22) Filed: Jan. 24, 2022

Related U.S. Application Data
(60) Provisional application No. 63/140,511, filed on Jan. 22, 2021.

(51) Int. Cl. A61M 5/168; A61M 5/00

(52) U.S. Cl. CPC A61M 5/16836; A61N 2005/1021; A61M 2205/3317; A61M 5/007

(57) ABSTRACT

A method of monitoring a fluid injection procedure is provided. The method includes: disposing a sensor on a catheter, where the sensor is in proximity to a tip of the catheter; inserting at least the tip of the catheter into a patient; delivering a fluid to a location within the patient via the tip of the catheter; and automatically monitoring a sensor signal from the sensor while the fluid is being delivered. Reflux end-point detection using an electrical impedance sensor has been demonstrated in a phantom. Applications include embolotherapy and angiography.

### CROSS REFERENCE TO RELATED APPLICATIONS

This application claims priority from U.S. Provisional Patent Application 63/140,511 filed Jan. 22, 2021, which is incorporated herein by reference.

### GOVERNMENT SPONSORSHIP

This invention was made with Government support under contract TR003142 awarded by the National Institutes of Health. The Government has certain rights in the invention.

### FIELD OF THE INVENTION

This invention relates to monitoring of fluid injection procedures performed with a catheter.

### BACKGROUND

In conventional medical practice, X-rays are required for angiography and monitoring of embolic material delivery through angiography catheters (to block blood flow to tumors or organs). If contrast or embolic material are injected at a rate that is too high, the contrast or embolic material will “reflux” backward around the catheter into anatomic regions or organs other than the ones intended. This is a problem because it can lead to embolic material traveling to healthy organs, and potentially damaging them (a phenomenon known as “off target embolization”). Similarly, reflux may occur with contrast material delivery for diagnostic angiography, in which case it may lead to contrast material highlighting other organs and causing diagnostic confusion, potentially missing tumors or vascular injuries from the desired vessels and potentially requiring repeat angiography. To avoid these problems, angiographers watch the delivery of embolic material using X-rays. However, X-rays are ionizing radiation that are dangerous for the patient and the physicians performing the procedure. Therefore, a critical problem exists that angiography and embolization require X-rays for monitoring.

### SUMMARY

To solve this problem, we have embedded an electrical impedance sensor just proximal to the tip of the angiography catheter. The sensor detects electrical impedance changes in injected contrast or embolic material, allowing embolization to be monitored without reliance on X-rays.

An exemplary embodiment has an electrical impedance sensor just proximal to the tip of an angiography catheter. When nothing is being injected through the catheter, the sensor is surrounded by blood, which has a characteristic electrical impedance range. When contrast and/or embolic material are injected at a low rate and there is no reflux, the sensor will still be surrounded by blood and will reflect the impedance measurement of blood. This condition is termed forward flow of fluid, whereby blood carries the injected fluid forward. When contrast and/or embolic material are injected at an excessively high rate, or embolization is nearing completion, the sensor will become surrounded by contrast material and/or embolic material and the impedance measurement will change. Specifically, if the impedance measurement changes to a value equal to that of contrast material and/or embolic material, it would indicate reflux of the injected material around the catheter. If the impedance measurement changes to a value intermediate between blood and the injected material and this value remains steady, this would indicate stasis. In the field of angiography, there is a concept of ‘near stasis,’ which represents slowing of the blood going to an organ but not to the degree of stasis. For some embolization procedures, some angiographers use near stasis as an end-point of treatment. In the context of impedance sensing described here, near stasis could be detected as a change in the impedance measurement to a value intermediate between blood and the injected material which changes back to blood at a rate determined by the residual flow of blood into the organ of interest. This rate could provide a quantitative metric of the degree of near stasis.

The sensor could embody a variety of 2, 3, or 4 wire electrical impedance measurement techniques, but in present experiments it is a two wire technique for easier fabrication. The sensing element includes two electrodes, which in current form are composed of conductive epoxy on top of wire leads, though conductive swaged marker bands bonded to wire leads or wrapped wire electrodes are an alternative. The wires run down the shaft of the catheter to the catheter hub, where they separate into leads that connect to the sensing circuitry. The sensing circuitry employs transimpedance measurement techniques with or without lock-in amplification. In current form, the circuitry sends values through wired connection or wirelessly to a computer that is able to show the impedance measurements as a continuous real-time graph.

For embolization, the reflux-sensing catheter promises to decrease the amount of radiation required for embolization, and to potentially eliminate off-target embolization. Decreased radiation would allow safer angiography for both the patient and physician, potentially also allowing the targeting of more lesions in a single treatment session (which is often limited by maximum permissible radiation to avoid negative deterministic of radiation). By eliminating off-target embolization, the reflux sensing catheter will preserve the health of adjacent organs.

For diagnostic angiography, the reflux-sensing catheter promises to avoid excessive contrast agent injection which create poor or nondiagnostic angiographic pictures. A common scenario is that a catheter ejects out of the target vessel due to excessive injection force. We envision a closed-loop control system enabled by a sensing catheter, whereby if reflux is sensed, the injection rate is automatically decreased. Currently, when this situation occurs, the injection is not stopped and the patient receives the full contrast agent load. As contrast agents can be damaging to the kidneys, it is desirable to avoid such a situation.

Also for both diagnostic angiography and embolization, we envision that the sensing element could be used to indicate that the catheter has perforated and/or dissected the blood vessel it is located in.

Advantages include:
decreased radiation required during angiography and embolization procedures, leading to enhanced safety for patient and physician;
safer embolization procedures with less risk of off-target embolization;
potential ability to target more tumors/organs if less radiation is required for each tumor/organ.

### BRIEF DESCRIPTION OF THE DRAWINGS

FIGS. 1A-B show an example of conventional X-ray monitoring of embolization therapy.

FIGS. 2A-C schematically show operation of an exemplary monitoring method according to an embodiment of the invention.

FIGS. 3A-C schematically show monitoring data obtained from an embolization phantom.

FIG. 4 shows electrical impedance vs. frequency for several relevant fluids.

### DETAILED DESCRIPTION

Embolization through angiography catheters has been a revolutionary targeted treatment, providing a minimally-invasive substitute for morbid surgical procedures and enabling therapies previously not possible. As part of the embolization process, small particles and fluids are delivered to organs through angiography catheters, with the intention of stopping bleeding in trauma, blocking blood flow to tumors, delivering chemotherapy to tumors, or decreasing blood flow to benign yet debilitating conditions like uterine fibroids or enlarged prostates. The applications of embolization are widespread, with indications continuing to expand.

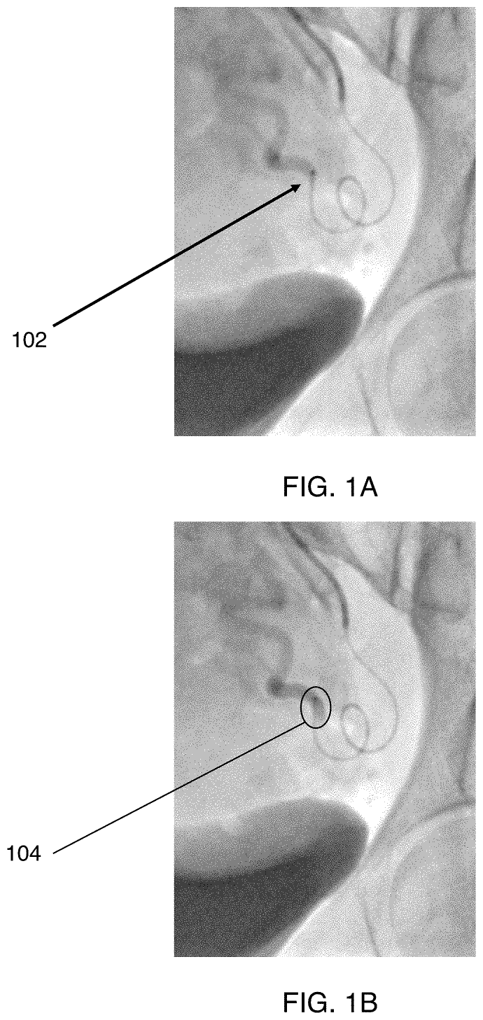

However, transcatheter embolization has a critical weakness: it requires X-ray imaging to assess the progress and end-points of embolization. X-rays are ionizing radiation, subjecting patients and treating physicians to DNA damage and the consequent dangers of radiation (skin injury, cataracts, cancer). Physicians performing embolization therefore must wear heavy protective equipment, which lead to high rates of occupational injury. Alarmingly, despite protection and radiation dosimetry, recent studies are showing that interventionalists have higher rates of chromosomal damage. Aside from the concerns of radiation safety, most embolic material cannot be directly visualized on X-ray. Thus, embolic material is typically suspended in iodinated contrast media so its delivery can be monitored with X-ray, but visualization remains challenging, especially in larger patients. X-rays are therefore an indirect and imperfect means of monitoring embolic delivery and off-target embolization. Moreover, patients with kidney failure or prior contrast-reaction cannot receive iodinated contrast media. FIGS. 1A-B show an example of conventional X-ray monitoring of embolization therapy. FIG. 1A is an X-ray image showing no reflux near catheter tip 102. FIG. 1B is an X-ray image showing reflux 104 around the catheter tip. As is apparent from these images, it can be challenging to accurately monitor the procedure.

To solve the reliance of embolization monitoring on dangerous X-rays, we integrate electrical impedance sensors at the tip of the angiography catheter that can sense the progress of embolization without the need for X-rays. The implementation of the aforementioned sensing angiographic catheter prototype involved attaching fine wire leads to the catheter with silver epoxy, which would be too fragile and too bulky for clinical use. Therefore, subsequent prototypes were created using thin-film metal electrodes via micro/nanofabrication techniques and are preferred due to their low profile, facilitating navigation through blood vessels.

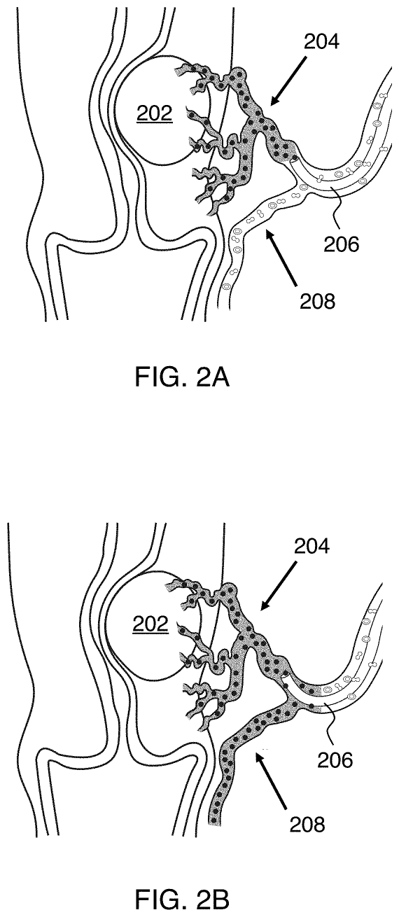

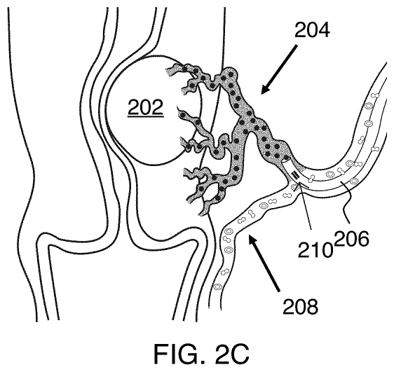

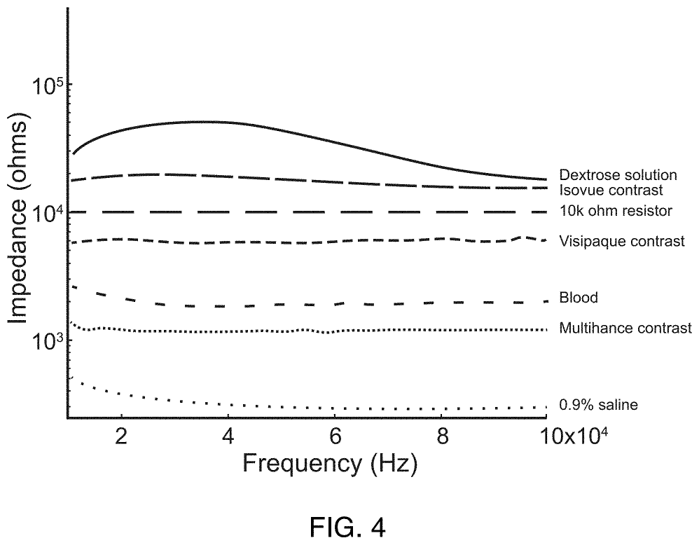

Preliminary data confirms that electrical impedance sensing at the catheter tip is a feasible approach to replacing X-ray monitoring of embolization. First, the electrical impedance spectra of several fluids regularly encountered during embolization were measured (FIG. 4). Blood exhibited an impedance spectrum very different from the carrier media often used for embolic delivery (nonionic iodine contrast or dextrose solution), meaning that when the carrier medium becomes static as the target organ fills with embolic, the carrier medium will displace blood and the sensor will register a different impedance value (FIGS. 2A-C).

Here FIG. 2A shows delivery of embolization material to blood vessels 204 feeding a mass 202 using a catheter 206. FIG. 2B shows the undesirable reflux condition, where too much embolization material is provided and the excess gets into blood vessel 208 that should not be embolized. FIG. 2C shows the idea of end-point detection, where a sensor 210 is used to determine when the embolization material reaches the sensor in order to provide a suitable signal for ending the therapy.

Accordingly, an embodiment of the invention is a method of monitoring a fluid injection procedure, where the method includes: disposing a sensor on a catheter, where the sensor is in proximity to a tip of the catheter; inserting at least the tip of the catheter into a patient; delivering a fluid to a location within the patient via the tip of the catheter; and automatically monitoring a sensor signal from the sensor while the fluid is being delivered.

Delivery of the fluid to the patient can be performed under closed loop control using the sensor signal as an input to a control system.

The tip of the catheter can be located within a blood vessel of the patient while the fluid is being delivered.

In such cases, the sensor signal can be further used to sense perforation or dissection of the blood vessel by the catheter.

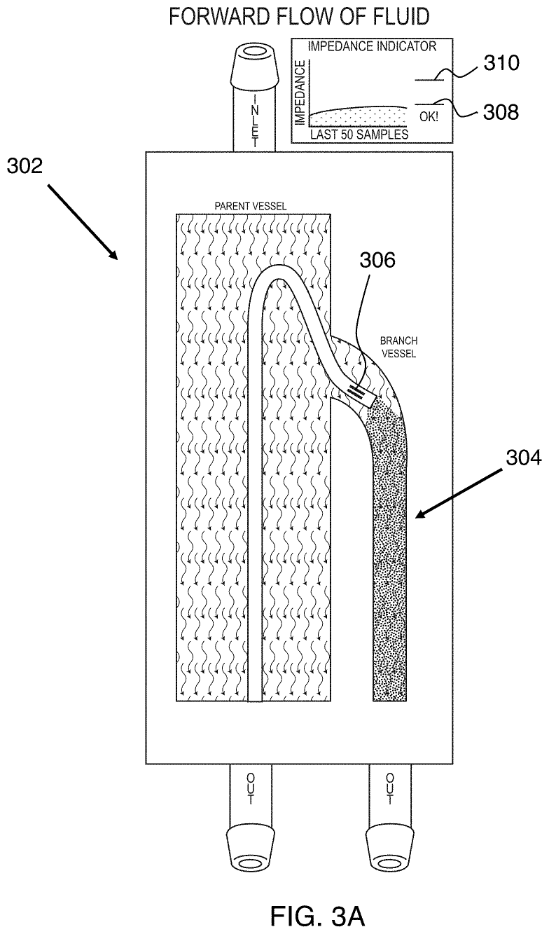

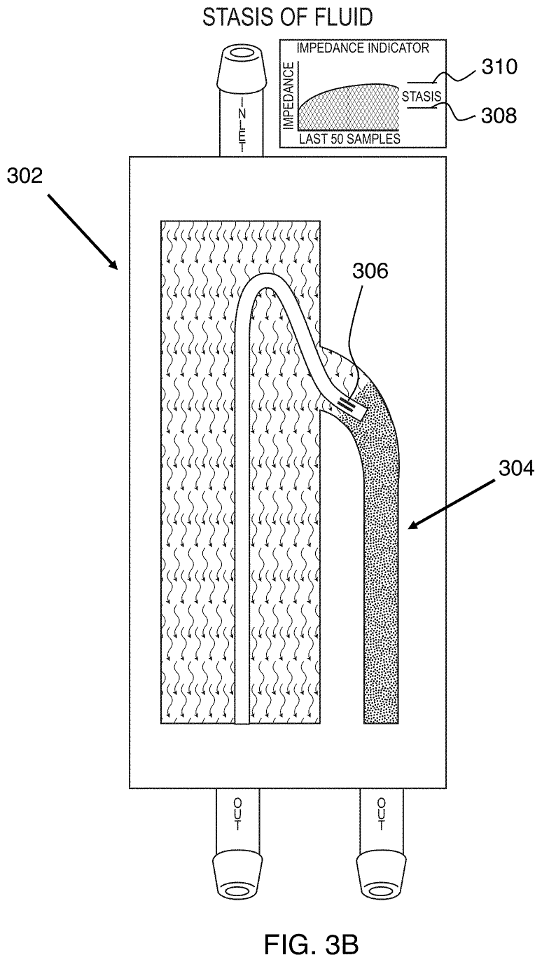

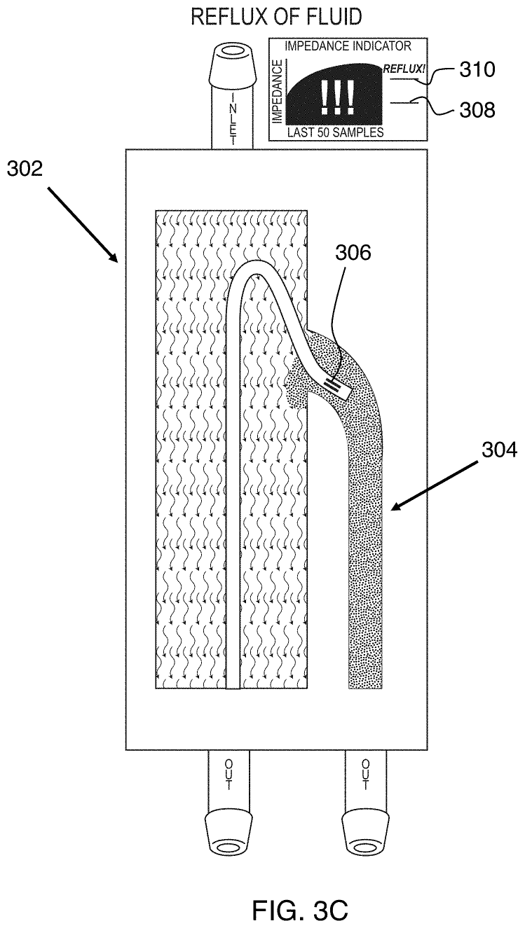

In phantom experiments, a 5 French (1.67 mm diameter) angiographic catheter was fitted with two electrodes near its tip to perform two-wire electrical impedance measurements in a flow phantom model (FIGS. 3A-C). Here 302 is the flow phantom, 304 is the modeled embolic carrier medium, 306 is the impedance sensor on the catheter, 308 is an impedance threshold separating forward flow from stasis or near-stasis, and 310 is an impedance threshold separating stasis from reflux.

Since saline has an electrical impedance value similar to blood, red colored normal saline was used as a blood mimicking fluid. Blue-colored 5% dextrose solution in water (a possible carrier medium for embolic material) was then injected through the angiography catheter into a target vessel of the phantom. As the vessel filled with the embolic carrier medium (dextrose solution) and started to reflux around the catheter tip and impedance sensor—which in the setting of embolization would indicate that delivery should be slowed—the electrical impedance value changed from that of the blood mimicking fluid to that of the embolic carrier medium (progression from FIG. 3A to FIG. 3C).

FIG. 4 shows electrical impedance vs. frequency for several relevant fluids.

Practice of the invention is not limited to end-point detection. These principles are applicable to any kind of automated monitoring of a fluid injection procedure, such as: detecting stasis of flow of the fluid, detecting near-stasis of flow of the fluid, detecting free flow of blood and detecting reflux of the fluid. Details of how these flow regimes are best defined will depend on specifics of the therapy being performed, but can be determined in each case by straightforward experimentation.

Practice of the invention is also not limited to electrical impedance sensing on the catheter. Suitable catheter sensors could be optical sensors, pressure sensors, temperature sensors, acoustic/ultrasound imaging sensors, and/or electrical sensors. Naturally, it is preferred for the sensor technology to be compatible with the catheter being used.

In cases where the sensor is an electrical impedance sensor the sensor signal can be at a predetermined frequency that is selected to provide an impedance contrast between the fluid and blood. Alternatively or in addition, the composition of the fluid can be selected to provide an impedance contrast between the fluid and blood. The sensor signal can also be an impedance spectrum at a predetermined frequency range. Such a multi-frequency signal can provide ‘fingerprints’ to more clearly distinguish the fluid being injected from blood. More generally, any sensor technology that can provide a multi-frequency sensor signal can be used to provide such fingerprints.

The preceding examples mainly relate to embolotherapy. There are several versions of this. Embolotherapy can involve delivering therapies including: liquid embolics (including cohesive embolics such as ethylene vinyl oxide, adhesive embolics such as glue, or sclerosants such as alcohol or sodium tetradecyl sulfate), particle embolics suspended in liquid, or gas embolics.

Practice of the invention is not limited to embolotherapy. It is applicable to any therapy where fluid (i.e. a liquid or a gas) is injected into a patient's body using a catheter. An important further application is angiography, as described above. Carbon dioxide gas is a possible contrast agent in angiography.

Further examples of fluid injection procedures include: intra-arterial injection of gene therapy, intra-arterial injection of cellular therapy, intra-arterial injection of immune therapy, intra-arterial injection of chemotherapy, and intra-arterial injection of radiation therapy.

1. A method of monitoring a fluid injection procedure, the method comprising:
disposing a sensor on a catheter, wherein the sensor is in proximity to a tip of the catheter;
inserting at least the tip of the catheter into a patient;
delivering a fluid to a location within the patient via the tip of the catheter; and
automatically monitoring a sensor signal from the sensor while the fluid is being delivered.

2. The method of claim 1, wherein the fluid injection procedure is angiography.

3. The method of claim 1, wherein the fluid injection procedure is embolotherapy.

4. The method of claim 1, wherein delivery of the fluid to the patient is performed under closed loop control using the sensor signal as an input to a control system.

5. The method of claim 1, wherein the tip of the catheter is located within a blood vessel of the patient while the fluid is being delivered.

6. The method of claim 5, wherein the sensor signal can be further used to sense perforation or dissection of the blood vessel by the catheter.

7. The method of claim 1, wherein the sensor is an electrical impedance sensor.

8. The method of claim 7, wherein the sensor signal is at a predetermined frequency, and wherein the predetermined frequency is selected to provide an impedance contrast between the fluid and blood.

9. The method of claim 7, wherein a composition of the fluid is selected to provide an impedance contrast between the fluid and blood.

10. The method of claim 7, wherein the sensor signal is an impedance spectrum at a predetermined frequency range.

11. The method of claim 1, wherein the sensor is selected from the group consisting of: optical sensors, pressure sensors, temperature sensors, acoustic/ultrasound imaging sensors, and electrical sensors.

12. The method of claim 1, wherein the monitoring includes detection of a condition selected from the group consisting of: stasis of flow of the fluid, near-stasis of flow of the fluid, free flow of blood and reflux of the fluid.

13. The method of claim 1, further comprising performing automatic end-point detection for the fluid injection procedure using the sensor signal.

14. The method of claim 1, wherein the fluid injection procedure is selected from the group consisting of: intra-arterial injection of gene therapy, intra-arterial injection of cellular therapy, intra-arterial injection of immune therapy, intra-arterial injection of chemotherapy, and intra-arterial injection of radiation therapy.
